(19) United States
(12) Patent Application Publication
(10) Pub. No.: US 2022/0202277 A1
(43) Pub. Date: Jun. 30, 2022

(54) STEERABLE MEDICAL DEVICE

(71) Applicant: Canon U.S.A., Inc., Melville, NY (US)

(72) Inventors: Showna Hsu-Hwa Chang, Arlington, MA (US); Takahisa Kato, Brookline, MA (US)

(21) Appl. No.: 17/552,286

(22) Filed: Dec. 15, 2021

Related U.S. Application Data
(60) Provisional application No. 63/149,963, filed on Feb. 16, 2021.
(60) Provisional application No. 63/132,374, filed on Dec. 30, 2020.

(51) Int. Cl. A61B 1/005; A61B 1/00

(52) U.S. Cl. CPC A61B 1/0057; A61B 1/00087; A61B 1/00103; A61B 1/00154

(57) ABSTRACT

A medical device, system, and methods of using the medical device and system are provided. Embodiments of the disclosure include a medical apparatus including a bendable body having a driving wire configured in the bendable body. A distal end of a break-out wire is attached to a proximal end of the driving wire where a distal guide tube guiding the driving wire, the distal tube ending before the break-out wire with a space. A resilient element abutting the driving wire along at least a portion of a longitudinal direction of the driving wire, and expands or contracts along the longitudinal direction of the driving wire. An actuator retracts and advances the driving wire via the break-out wire and maneuvers the bendable body, wherein the resilient element stays within the space between a proximal end of the distal guide tube and the distal end of the break-out wire.

### CROSS REFERENCE TO RELATED PATENT APPLICATIONS

This application claims priority from U.S. Provisional Patent Application No. 63/132,374 filed on Dec. 30, 2020, as well as U.S. Provisional Patent Application No. 63/149,963 filed on Feb. 16, 2021, both filed in the United States Patent and Trademark Office, the disclosures of which are incorporated herein in their entirety by reference.

### FIELD OF THE DISCLOSURE

The present disclosure relates generally to medical devices and, more particularly to a steerable device applicable to guide interventional tools and instruments, such as endoscopes and catheters, in medical procedures.

### BACKGROUND OF THE DISCLOSURE

United States Patent Application Publication No. 2020/0383670 describes a medical device having a bendable body for insertion into a patient. The medical device includes at least a first driving wire configured in the bendable body, a first break-out wire attached to the first driving wire, and a contracting guide that is movable against the first driving wire. The first break-out wire is connected to an actuator configured to maneuver the bendable body to guide the medical device through passages. The contracting guide may be a contractible coil parallel to the first driving wire, and situated to contact the first driving wire when a motor conveys pushing and pulling forces.

To avoid buckling the driving wire when the contracting guide contacts the driving wire, the contractible coil surrounds the driving wire and the diameter of the driving wire is converted to the diameter of the break-out wire to avoid buckling thin driving wires. U.S. Pub. No. 2020/0383670 does not, however, describe that the contractible coil covers the full stroke of the driving wires. Where the contractible coil does not cover the full stroke of the driving wires, there is a risk of buckling of the driving wires around the boundary of the driving wires with the break-out wires.

### SUMMARY

Embodiments of the present disclosure provide a medical apparatus comprises a bendable body having a driving wire configured in the bendable body, a break-out wire attached to the driving wire, wherein a distal end of the break-out wire is attached to a proximal end of the driving wire, a distal guide tube guiding the driving wire and ending before the break-out wire with a space; a resilient element abutting the driving wire along at least a portion of a longitudinal direction of the driving wire; and an actuator configured to retract and advance the driving wire via the break-out wire thereby maneuvering the bendable body. The resilient element expands or contracts along the longitudinal direction of the driving wire, and it stays within the space between a proximal end of the distal guide tube and the distal end of the break-out wire. In some embodiments, the length of the resilient element is at least as long as a length of the driving wire from a proximal end of the distal guide tube.

Embodiments of the present disclosure also provide a steerable medical device including a resilient element such as a spring and a driving wire, with a length of the spring being greater than a length of the driving wire. According to various embodiments, the spring is a contractible coil spring which covers the full stroke of the driving wire. The present disclosure thus reduces, or eliminates, the risk of buckling of the driving wire by providing a medical device which reduces uncovered stroke of the driving wire with the spring.

According to embodiments of the present disclosure, a medical apparatus comprises a bendable body having a driving wire configured in the bendable body, a break-out wire attached to the driving wire, wherein a distal end of the break-out wire is attached to a proximal end of the driving wire, a resilient element such as a spring surrounding the driving wire along at least a portion of a longitudinal direction of the driving wire, the spring being movable against the driving wire; and an actuator configured to retract and advance the driving wire via the break-out wire and configured to maneuver the bendable body, wherein the spring contracts along the longitudinal direction of the driving wire, and a length of the spring is at least as long as a length of the driving wire from a proximal end of a distal guide tube that guides the driving wire to the distal end of the break-out wire.

According to some embodiments, a medical apparatus comprises a bendable body, at least one driving wire attached to the bendable body, an actuator, a break-out wire guide, a spring, and at least one break-out wire. The bendable body has a centroid along a longitudinal direction, the centroid including a proximal end and a distal end. The at least one driving wire is attached to the bendable body and extends to the proximal end of the centroid through the bendable body with an offset distance from the centroid. The actuator pushes and pulls one of the driving wires, the break-out wire guide bridging between the proximal end of the driving wires and the actuator to transmit push and pull forces from the actuator to the driving wires. The spring contracts along the longitudinal direction, and the at least one break-out wire has a larger diameter than the driving wire. An eyelet in the break-out wire guide has a proximal area and distal area with different cross sectional sizes along the longitudinal direction, wherein the cross sectional size of the proximal area of the eyelets is larger than the cross sectional size of the distal area of the eyelet, and the driving wire goes through the eyelet from the distal area to the proximal area of the eyelet, and the break-out wire is attached to the proximal end of the driving wire and extends through the proximal area of the eyelet, and is attached to the actuator. Wherein the spring surrounds the driving wire in the proximal area of the eyelet, and is surrounded by the eyelets, and is contracted by the break-out wire while the actuator is pushing the driving wire. The natural length of the spring is longer than a length of the driving wire from the distal guide tube to the distal end of breakout wire at the most proximal operation position of tractor. In some embodiments, the spring is compressed at the operation position of tractor for the straight shape of the bendable body.

According to some embodiments, a medical apparatus comprises an anchored spring without pre-tension or compression. The natural length of the spring is the same as the length of the driving wire from the distal guide tube to the distal end of breakout wire at the operation position of tractor for the straight shape of the bendable body. A distal end of the spring is attached to the boundary of the distal and proximal guide tubes. And a proximal end of the spring is attached to the boundary of the driving and breakout wires.

According to some embodiments, the diameter of the breakout wire is larger than the diameter of the driving wire, thus further aiding in avoiding buckling of the medical apparatus. Further variants may include a tapered breakout wire and/or driving wire to assist in retaining structural integrity.

According to some embodiments, a medical apparatus comprises a bendable body having a compression resistance element to maintain the length of the bendable body against compression force. One or more of the driving wire, the break-out wire guide, the spring, and the break-out wire are detachably attached to actuator.

Further features of the present disclosure will become apparent from the following description of exemplary embodiments with reference to the attached drawings.

### BRIEF DESCRIPTION OF THE DRAWINGS

FIG. 1A depicts a side perspective view of a steerable medical device, according to one or more embodiments of the present disclosure.

FIG. 1B is a front perspective view of a steerable medical device, according to one or more embodiments of the present disclosure.

FIG. 2A provides a top perspective view of at least a portion of a steerable medical device, according to one or more embodiments of the present disclosure.

FIG. 2B provides a top perspective view of at least a portion of a steerable medical device, according to one or more embodiments of the present disclosure.

FIG. 3 details a side perspective view of at least a portion of a steerable medical device, according to one or more embodiments of the present disclosure.

FIGS. 4A, 4B, and 4C each depict a side perspective view of at least a portion of a steerable medical device, according to one or more embodiments of the present disclosure.

FIGS. 5A, 5B, and 5C each depict a side perspective view of at least a portion of a steerable medical device, according to one or more embodiments of the present disclosure.

Throughout the figures, the same reference numerals and characters, unless otherwise stated, are used to denote like features, elements, components or portions of the illustrated embodiments.

### DETAILED DESCRIPTION OF THE DISCLOSURE

Exemplary embodiments are described below with reference to the drawings. The following description and drawings are illustrative of the disclosure and are not to be construed as limiting the disclosure.

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

The present disclosure details a medical device capable of being steered for guidance through passages. More specifically, the subject medical device contains a central cavity, extending the length of the medical device, for accepting and advancing medical tools and devices including endoscopes, cameras, and catheters, and the ability to guide or maneuver the medical tool or device through passages.

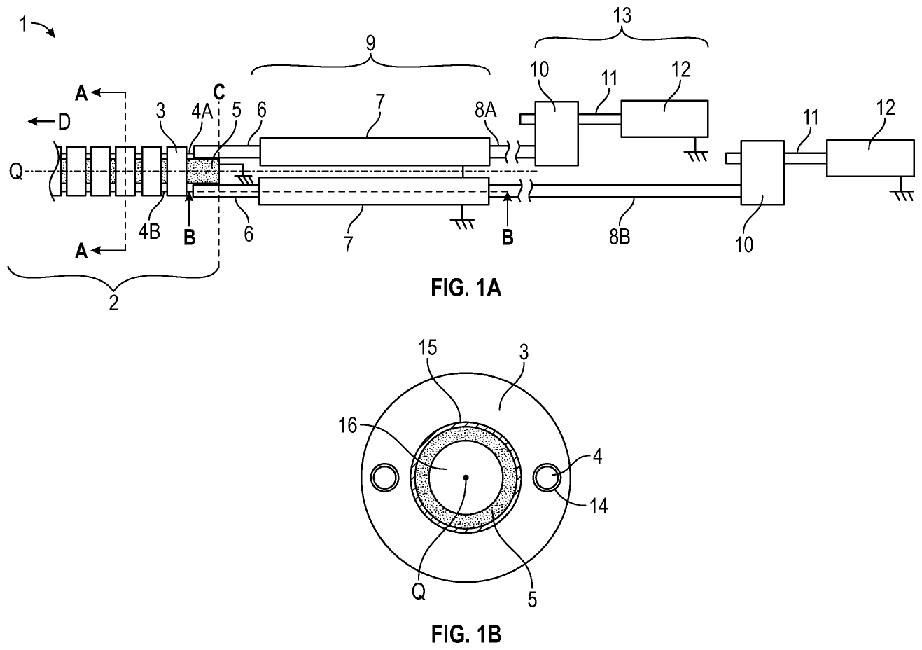

FIG. 1A provides a side perspective view of a steerable medical device 1, according to one or more embodiments of the present disclosure. The steerable medical device 1 comprises a bendable body 2, a break-out unit 9, and an actuator 13. The bendable body 2 comprises at least two guide rings 3, driven by at least two driving wires 4A and 4B, a flexible backbone 5 for supporting the guide rings 3, as well as a guide ring eyelet 14 (see FIG. 1B) for each one of the driving wires 4. The guide rings 3 are hollow in the center and are fixed on the backbone 5 with an adhesive layer 15 by a designed interval. The assembly of the guide rings 3 in combination with the backbone 5 creates a channel 16 for accepting a secondary tool. The bendable body 2 also defines a centroid Q as a center line of tube-like shape of the bendable body 2, with proximal end C closer to the break-out unit 9, and a distal end D farther away from the break-out unit 9. The backbone 5 may be mechanically grounded at the proximal end C and elastically steerable but is rigid along centroid Q.

FIG. 1B further provides a front cross-sectional perspective view of a steerable medical device 1, provided in FIG. 1A. Accordingly, the cross section view of the bendable body 2 at cross section A-A (see FIG. 1A) more clearly details a guide ring 3 containing at least two guide ring eyelets 14 for accepting the at least two driving wires 4. FIG. 1B further details an adhesive 15, the channel 16 for accepting a secondary tool, as well as further defining the centroid Q.

Returning to FIG. 1A, the guide rings 3 may be fixed on the backbone 5 with the adhesive 15, or other means for affixing the rings 3 to the backbone 5, and may hold the driving wires 4A and 4B in the guide ring eyelets 14, while the driving wires 4A and 4B freely slide along the guide ring eyelets 14. Between adjacent guide rings 3, the driving wires 4A and 4B stand without any mechanical support structures. The space between guide rings 3 allows for manipulation of one guide ring 3 with respect to a second guide ring 3, thus leading to steering of the steerable medical device 1.

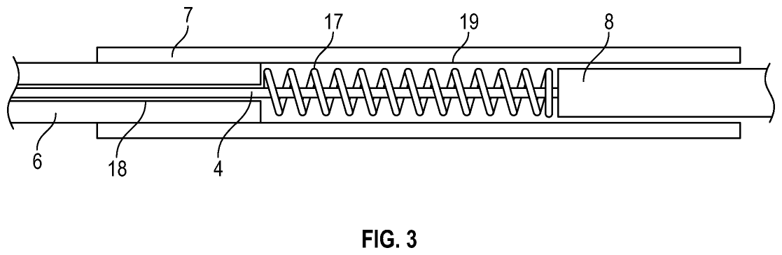

The break-out unit 9 comprises a distal guide tube 6, a proximal guide tube 7, at least two break-out wires 8A and 8B, and a spring 17 (see FIG. 3). The proximal guide tube 7 is mechanically grounded. Also, the distal guide tube 6 is fixed on the inner wall of the proximal guide tube 7. These distal and proximal guide tubes 6 and 7 create the distal and the proximal areas of eyelets, 18 and 19, respectively (see FIG. 3).

FIG. 1A further illustrates the actuator 13, which may be configured near the patient and is mechanically connected to the bendable body 2 via the break-out unit 9. The actuator 13 comprises a tractor 10 which is connected to the break-out wire 8, as well as a lead screw 11 and motor 12, for advancing and retracting the break-out wire 8 along the Q axis. The break-out wire 8 in this interval travels through free space and is kept straight. The tractor 10 may have threaded holes and the lead screw 11 may engage with these threaded holes. The lead screw 11 is attached to the motor 12.

The two motors 12 shown in FIG. 1A can rotate the lead screws 11 and independently push or pull the driving wires 4A and 4B via the lead screws 11 and the break-out wires 8A and 8B.

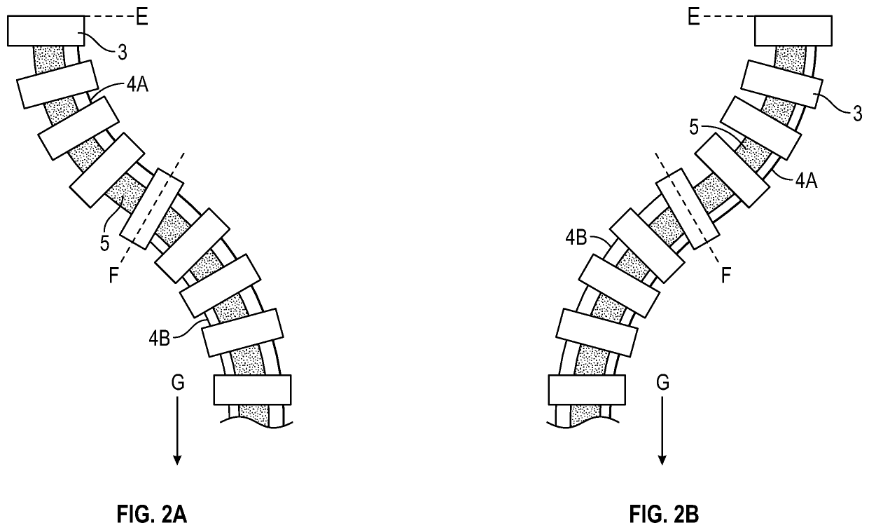

As depicted in FIGS. 2A and 2B, the driving wires 4A and 4B, at their respective distal ends, are fixed to the guide rings 3 at different positions. Specifically, the driving wire 4A is fixed to a first guide ring 3 at position E (i.e., the distal end of the bendable body 2), while the driving wire 4B is fixed to a second guide ring 3 at position F. The remainder of each driving wire 4A and 4B extends freely through the respective guide ring eyelets 14 until they reach the break-out unit 9. With these termination positions E and F, the bendable body 2 is divided into two bending sections, which is a substantial bending area that comprises an assembly of the backbone 5, the driving wire 4A or 4B and all the guide rings 3 in this area. In FIGS. 2A and 2B we observe two different enactments of the subject steerable medical instrument 1, wherein FIG. 2A portrays a left arcing S bend, accomplished by first retracting the driving wire 4B, followed by retracting the driving wire 4A. The right arcing S bend in FIG. 2B is accomplished by first advancing the driving wire 4B, followed by advancing the driving wire 4A. Stated another way, when the driving wire 4A is retracted by the motor 12, a bending section between position E and F bends to the direction of the driving wire 4A (FIG. 2A). Also, when the driving wire 4A is advanced, the bending section between position E and F can bend to the opposite direction (FIG. 2B). In the same way, when the driving wire 4B is retracted, a bending section between position F and G bends to the direction of the driving wire 4B, and when advanced, the bending section bends to the opposite direction. At the bending section between position F and G, the bending moment from the driving wires 4A and 4B interfere with each other, but by choosing appropriate actuation forces for the driving wires 4A and 4B, the bending section between position F and G can be bent independently. Consequently, by using two or more driving wires, two or more corresponding bending sections are independently bendable.

FIG. 3 provides a side cross-sectional perspective view of a steerable medical device 1, provided in FIG. 1A. Specifically, FIG. 3 provides a cross-sectional view at line B-B in FIG. 1A. In FIG. 3, the proximal end of the driving wire 4 is connected to the break-out unit 9. The distal area of the eyelet 18 mechanically guides the driving wire 4 along centroid Q. The distal area of the eyelet 18 has a smaller diameter than the proximal area of the eyelet 19. The proximal area of the eyelet 19 includes the driving wire 4 and the break-out wire 8 and mechanically guides the break-out wire 8 along centroid Q. The proximal area of the eyelet 19 includes the spring 17, exemplified herein as a coil spring, for guiding the driving wire 4 along centroid Q. In some embodiments, one end of the spring 17 touches the distal guide tube 6 and the other end of the spring 17 touches the break-out wire 8. Between the distal guide tube 6 and the break-out wire 8, there is a space that contains the resilient element (the spring 17). The spring 17 contracts when the driving wire 4 is pushed by the break-out wire 8. While the spring 17 is compressing, the spring 17 acts to guide the driving wire 4 with a substantial identical inner diameter compared to the diameter before the spring 17 is compressed. Therefore, during a pushing operation of the break-out wire 8, the driving wire 4 can avoid buckling.

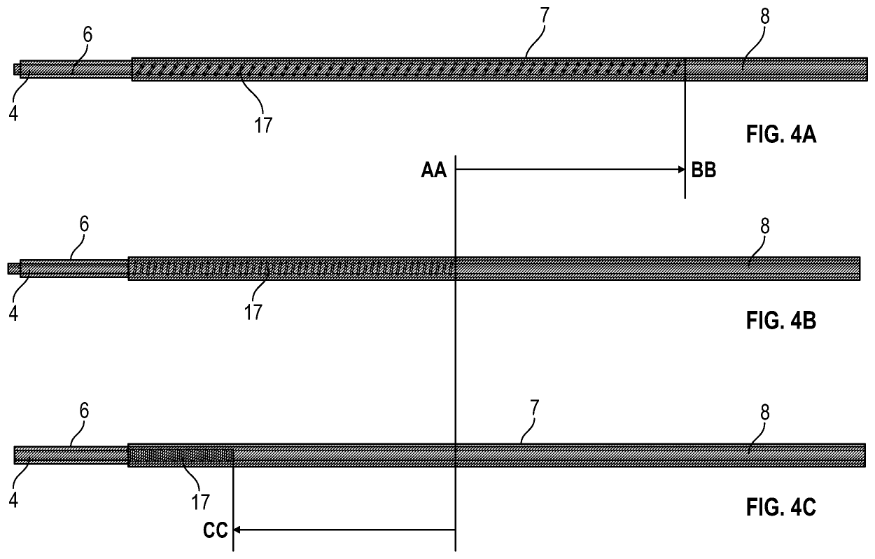

FIGS. 4A, 4B and 4C are schematics detailing the coverage of the spring 17 during bending operations by the motors. Specifically, the spring 17 in this embodiment is compressed when the bendable body 2 has a straight shape. However, in other embodiments, different types of springs or other resilient elements are sued instead of this spring.

Position AA in FIG. 4B signifies the position of the boundary of driving wire 4 and break-out wire 8, when the bendable body 2 has the straight shape. Therefore, this position corresponds to the center position in the full stroke of the driving wires for full range of bending of the bendable body 2. On the other hand, position BB in FIG. 4A signifies the position of the boundary of driving wire 4 and break-out wire 8 at the edge of the stroke on the pulling side.

The spring 17 has a neutral length without any tension and compression at position BB (FIG. 4A). Therefore, the spring 17 covers driving wire 4 at this position to prevent buckling of driving wire 4.

When the driving wire 4 is pushed from position BB to AA, the spring 17 is compressed (FIG. 4B). During this motion, the spring 17 continues to cover the driving wire 4 to prevent any kinking.

When the driving wire 4 is further pushed beyond position AA to CC (FIG. 4C), the spring 17 is further compressed and continues to cover the driving wire 4.

Accordingly, in this embodiment, the spring 17 covers the driving wire 4 from position BB to position CC, which is the full range of the required stroke, and can prevent the buckling of the driving wire 4 whenever the driving wire 4 is pushed.

Specifically, with this embodiment, spring 17 can be assembled in the break-out unit 9 without bonding/attaching to the other part. This embodiment simplifies the manufacturing and structure of break-out unit 9, with miniaturized size and affordable cost. Moreover, no bonding elements results in greater durability of the break-out unit 9.

Also, since the spring 17 is not compressed at position BB, spring 17 is subjected to minimal compressed forces through full range of stroke during operation. This feature has advantages in reducing the driving force by the amount of compressed force for actuator 13.

Moreover, when the actuator 13 measures force on the driving wire 4, the compressed force to the spring 17 would be systematic error factor. By minimizing the compressed force, we can also reduce the systematic error for wire force measurement from the actuator 13.

In yet another embodiment, the spring 17 may have a compressed condition at position BB. With this design, the spring 17 is always compressed from position BB to position CC. This design presents an advantage in keeping spring 17 from rattling by having compressed force everywhere from position BB to position CC.

As described above, according to the exemplary embodiment described with reference to FIGS. 4A to 4C, the spring 17 covers the driving wire 4 from position BB to position CC, which is the full range of the required stroke. Accordingly, the spring 17 can prevent the buckling of the driving wire 4 whenever the driving wire 4 is pushed or pulled. According to the present embodiment, the spring 17 can be assembled in the break-out unit 9 without bonding or attachment to any other parts of the medical device. This simplifies the manufacturing and structure of the break-out unit 9 with miniaturized size and affordable cost. Moreover, no bonding part is beneficial for durability of the break-out unit 9.

Another embodiment of the present disclosure has a similar configuration of the steerable medical device 1 in FIG. 3, however one end of spring 17 is connected about the distal and proximal guide tubes 6 and 7. Alternatively or in addition, the opposite end of spring 17 may be connected to about the driving wire 4 and the break-out wire 8.

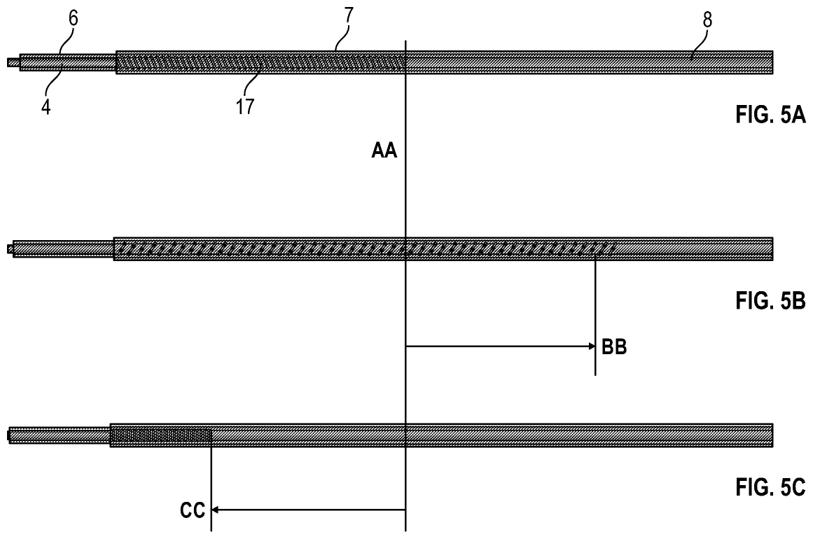

FIGS. 5A, 5B, and 5C are the schematics to explain the coverage of the spring 17 during bending operation by motors under this embodiment.

As seen in FIGS. 5A-5C, one end of the spring 17 is attached to the proximal end of the distal guide tube 6 while the other end of the spring 17 is attached to the distal end of the break-out wire 8. Then, at position AA, the spring 17 has natural length without compression and tension forces (FIG. 5A). When driving wire 4 is pulled from position AA and reaches position BB (FIG. 5B), the spring 17 is also pulled since both ends are attached, thus creating tension in the spring 17.

When the driving wire 4 is pushed from position AA and reaches position CC (FIG. 5C), the spring 17 is also pushed since both ends are attached, creating compression in the spring 17. Accordingly, the spring 17 can cover driving wire 4 from position BB to position CC, which is the full range of the required stroke, and can prevent the buckling of driving wire 4 whenever driving wire 4 is pushed.

Specifically, in this embodiment, the absolute magnitude of compression and tension force to the spring 17 can be minimized. This allows minimizing the additional force required to manipulate the actuator 13.

Moreover, when the actuator 13 will measure force on driving wire 4, those compressed and tension forces to the spring 17 would be systematic error factor. By minimizing the absolute magnitude of those forces with this embodiment, we can also reduce the systematic error for wire force measurement from the actuator 13.

The distal end of spring 17 can be attached to the proximal end of distal guide tube 6. In addition, the proximal end of spring 17 can be attached to the proximal end of the driving wire 4.

As described above, according to the exemplary embodiment described with reference to FIGS. 5A to 5C, the spring 17 covers the driving wire 4 from position BB to position CC, which is the full range of the required stroke. Accordingly, the spring 17 can prevent the buckling of the driving wire 4 whenever the driving wire 4 is pushed or pulled, minimizing the absolute magnitude of compression and tension force on the spring 17, which allows minimizing the additional required force for manipulating the actuator 13. Moreover, when the actuator 13 will measure force on the driving wire 4, those compressed and tension forces to spring 17 would be systematic error factor. By minimizing the absolute magnitude of those forces with this embodiment, we can also reduce the systematic error for wire force measurement from actuator 13.

A third embodiment of the present disclosure has a similar configuration of the steerable medical device 1 detailed in FIG. 3. However, the break-out wire 8 is detachably attached to the tractor 10. Accordingly, an assembly of the bendable body 2 and break-out unit 9 is detachable from the actuator 13. This configuration provides advantages in allowing and end user to exchange the bendable body 2 from actuator 13, if necessary. For example, when bendable body 2 is broken, or has a different design/trajectory for target anatomy or location. Specifically, when the bendable body 2 is a one-time-use disposable device, this configuration allows for use of the actuator 13 as a reusable unit, leading to a savings in cost.

While this configuration has aforementioned advantages, this configuration adds a new requirement to maintain length and shape of the bendable body 2 while the break-out unit 9 is detached from actuator 13.

When the bendable body 2 and break-out unit 9 are detached from actuator 13, the bendable body 2 would be subjected to the following two factors to change its length and shape.

The first factor is the force from spring 17 in the break-out unit 9. Since the break-out wire 8 is not connected to the tractor 10, the spring 17 would convey pulling or pushing forces to the bendable body 2 if the spring 17 is compressed or tensioned at position AA. In this case, the bendable body 2 is compressed or tensioned by the compressed or tensioned spring 17, respectively.

The second factor is the force from outside. Since the break-out wire 8 is not connected to the tractor 10, the break-out wire 8 is relatively free to move. In this instance, when the bendable body 2 contacts some object in the environment, the bendable body 2 is bent by the outside force.

According to some embodiments, the following designs are effective to maintain the length and shape of bendable body 2 when detached from actuator 13.

Design 1. Break-Out Unit of First Embodiment with Compression Resistance Element in Bendable Body 2

Break-out unit 9 of the first embodiment has a compressed spring 17 at position AA. In this configuration, when the bendable body 2 is detached from actuator 13, the bendable body 2 is subjected to a compression force by the compressed spring 17. The bendable body 2 has a compression resistance element, which can be backbone 5 in FIGS. 1A, 1B, 2A, and 2B. The backbone 5 can be constructed of a rigid metal tube with laser cuts for providing bending flexibility, or alternatively constructed of rods and/or braided, or further include reinforced elastomer tubing for rigidity and flexibility.

The compression resistance element needs to cope with the compression force from spring 17, so as to retain the spring 17 within the wanted tolerance values of compression and expansion.

According to some embodiments, a catheter may be the compression resistance element in bendable body 2. The catheter may be designed such that the inner and outer tubes in the bending section can be the compression resistance element. Especially, when the inner tube includes metal braid structure, the inner tube can be reinforced in terms of compression force.

Design 2. Break-Out Unit of Second Embodiment with any Bendable Body 2

Since the break-out unit 9 of the second embodiment doesn't have a compression and tension force on the spring 17 at position AA, the break-out unit 9 of the second embodiment would maintain the length of bendable body 2.

Moreover, when the driving wire 4 moves from position AA toward position BB or position CC, the spring 17 generates a force to return to position AA. Therefore, break-out unit 9 of the second embodiment would also maintain the shape of bendable body 2.

Design 3. Break-Out Unit of Second Embodiment with Compressed or Tensioned Spring 17 with Compression or Tension Resistance Element in Bendable Body 2

Break-out unit 9 of the second embodiment can have a compressed or tensioned spring 17 at position AA by design of spring 17. The degree of compression or tensioning with this configuration would have a wider range than the break-out unit 9 detailed in the first embodiment, since the first embodiment requires that the maximum length of spring without tension should be larger than the length to cover driving wire 4 at position BB. Therefore, the break-out unit 9 of the second embodiment can have optimal compression or tension force for bendable body 2.

In this design, with the compressed spring 17 at position AA, the bendable body 2 has compression resistance element, which is the same as Design 1. Accordingly, when the bendable body 2 is detached from actuator 13, the bendable body 2 would be subjected to tension forces by tensioned spring 17. The bendable body 2 has a tension resistance element, which has a force stronger than tension or compression force of the spring 17, thus resisting the springs 17 force. This tension resistance element can be backbone 5 in FIGS. 1A, 1B, 2A, and 2B. The backbone 5 can be configured by tube made of elastomer, for example urethane and PEBAX®, ropes or wires. Generally, a long object with thin diameter just like bendable body 2 is stronger with tension force than compression force. This combination gives the simplest structure for bendable body 2.

According to some embodiments, a catheter may be the tension resistance element in bendable body 2. The catheter may be designed such that the inner and outer tubes in the bending section can be the tension resistance element.

As described above, the third embodiment provides a configuration which allows exchanging the bendable body 2 from the actuator 13 if it is necessary. For example, when the bendable body 2 is broken, or has different design/trajectory for target anatomy or location. Specifically, when the bendable body 2 is intended for one-time-use, this configuration allows using the actuator 13 as a reusable device. Three design embodiments under the third embodiment maintain the length and shape of the bendable body 2 when it is detached from the actuator 13. This can guarantee the designed function of the bendable body 2 in the actual implementation of the detachably attached bendable body 2 to actuator 13.

While the present disclosure has been described with reference to exemplary embodiments, it is to be understood that the present disclosure is not limited to the exemplary embodiments described.

What is claimed is:

1. A medical apparatus comprising:
a bendable body having a driving wire configured in the bendable body;
a break-out wire attached to the driving wire, wherein a distal end of the break-out wire is attached to a proximal end of the driving wire;
a distal guide tube guiding the driving wire and ending before the break-out wire with a space;
a resilient element abutting the driving wire along at least a portion of a longitudinal direction of the driving wire; and
an actuator configured to retract and advance the driving wire via the break-out wire thereby maneuvering the bendable body,
wherein the resilient element expands or contracts along the longitudinal direction of the driving wire, and
wherein the resilient element stays within the space between a proximal end of the distal guide tube and the distal end of the break-out wire.

2. The medical apparatus according to claim 1, wherein a length of the resilient element is at least as long as a length of the driving wire from a proximal end of the distal guide tube.

3. The medical apparatus according to claim 1, wherein a distal end of the resilient element is attached to the proximal end of the distal guide tube, and the proximal end of the resilient element is attached to the distal end of the break-out wire.

4. The medical apparatus according to claim 1, wherein the break-out wire is detachably attached to the actuator.

5. The medical apparatus according to claim 1, wherein the bendable body comprises a compression resistant element that is detachable from the actuator.

6. The medical apparatus according to claim 1, wherein the bendable body comprises a tension resistant element that is detachable from the actuator.

7. The medical apparatus according to claim 1, wherein the bendable body comprises a single-use medical device.

8. The medical apparatus according to claim 1, wherein the bendable body is configured to accept various surgical tools selected from the group consisting of a biopsy tool, an endoscope, a cutting tool, a slicing tool, a light, and combinations therefrom.

9. The medical apparatus according to claim 1, wherein the driving wire extends to a distal end of the bendable body and is offset from a center line of the bendable body.

10. The medical apparatus according to claim 1, further comprising:
a break-out unit for housing the break-out wire, wherein the break-out unit comprises a guide tube to guide the break-out wire.

11. The medical apparatus according to claim 1, further comprising:
a second driving wire configured in the bendable body; and
a second break-out wire attached to the second driving wire, wherein the second break-out wire is in communication with the actuator.

12. The medical apparatus according to claim 11, wherein the second driving wire partially extends into the bendable body and is offset from a center line of the bendable body.

13. The medical apparatus according to claim 11, wherein the actuator is configured to retract and advance the second driving wire via the second break-out wire.

14. The medical apparatus according to claim 11, wherein the second driving wire is configured in a different position than the driving wire, with respect to the bendable body.

15. The medical apparatus according to claim 11, wherein the bendable body comprises:
at least two guide rings, wherein the at least two guide rings are aligned parallel to one another and have a distance between them, and each of the at least two guide rings comprises at least one eyelet configured to at least accept the driving wire or the second driving wire.

16. The medical apparatus according to claim 1, wherein the resilient element is a coil spring.

17. The medical apparatus according to claim 1, wherein the resilient element comprises at least two coil springs.

18. The medical apparatus according to claim 1, wherein a diameter of the break-out wire is larger than a diameter of the driving wire.

19. The medical apparatus according to claim 1, wherein the break-out wire is tapered.

20. The medical apparatus according to claim 1, wherein the driving wire wire is tapered.
